(19) United States
(12) Patent Application Publication
(10) Pub. No.: US 2014/0261418 A1
(43) Pub. Date: Sep. 18, 2014

(54) Preheating device of the pipeline between air pressure source and humidifier

(71) Applicant: Neo HUANG, Taoyuan Hsien (TW)

(72) Inventor: Neo HUANG, Taoyuan Hsien (TW)

(21) Appl. No.: 13/845,611

(22) Filed: Mar. 18, 2013

(51) Int. Cl. A61M 16/10; A61M 16/00; A61M 16/08; A61M 16/16

(52) U.S. Cl. CPC A61M 16/109; A61M 16/16; A61M 16/0066; A61M 16/0875
USPC 128/203.26

(57) ABSTRACT

A preheating device is installed to the pipeline connecting between an air pressure source and a humidifier to increase the temperature of the gas entering the humidifier and to reduce the relative humidity. The gas can fully mix with vapor generated by a heated water carrier within a short time, thereby preventing the humidifier from excessively outputting condensed water.

### BACKGROUND OF THE INVENTION

1. Field of Invention

The invention relates to a respiratory humidification system and, in particular, to a preheating device interposed between an air pressure source and a humidifier in order to preventing the humidifier from excessively outputting condensed water.

2. Related Art

The humidification apparatus or respiratory humidification system is common medical equipment for the hospital emergency room and intensive care unit. When a patient is unable to breathe, or can breathe on his own but cannot reach the concentration of oxygen needed by the body, the humidification apparatus is then required to sustain life. This applies in particular to patients with lung diseases, such as SARS, H1N1, or patients who have experienced a lung injury.

FIG. 1 shows the functional principle of a humidification apparatus. A gas supply means, commonly known as air pressure source, is built in with a fan or compressor (not shown). The air thus generated is sent, as indicated by the arrow, via a conduit 2 to a humidifier 3. The humidifier comprises a humidification chamber 31 and a heater plate 32. The heater plate 22 heats the water carrier in a humidification chamber 21, so that the gas provided by the air pressure source takes away the vapor generated by the heated water carrier. The humidified gas is then delivered through an inspiratory conduit 4 to the patient 5 at the best breathing temperature and humidity.

Before the vapor produced by the heated water carrier of the humidifier reaches the patient, the vapor is easily condensed into water due to the influences of ambient temperature, airflow, and the airflow temperature. Therefore, the temperature and vapor are dissipated 1 in the intake duct by the phenomenon commonly known as condensation.

In order to reduce the condensation, a general approach is to provide heating wire means 41 in the intake duct in order to maintain the temperature.

Another approach is to increase the heating power of the humidifier to compensate for the loss of temperature and water vapor. As the heating power gets higher, the loss of temperature and water vapor also increases simultaneously. This results in more water condensate in the intake duct. The most serious situation may lead to the clogging of the intake duct.

Besides, the temperature of the gas generated by the gas supply means is far less than the heating temperature of the humidifier. The time for the gas to pass by the water carrier is too short. Not only can the gas not be sufficiently mixed with water vapor, there is also not enough time to heat up the cold air. Thus, the water vapor condenses in the intake duct before reaching the patient end.

In order to improve the condensation problem of the intake duct, the renowned New Zealand manufacturer Fisher & Paykels Healthcare Limited has proposed the following U.S. patents: U.S. Pat. Nos. 8,206,337, 8,037,882, 7,958,891, 7,559,324, 7,263,994, 7,146,979, 7,043,979, 6,918,389, 6,662,802, RE 40,806, and RE 39,724. However, the above-mentioned solutions have higher costs because either the pipeline uses some high-tech materials or pipeline structure is too complicated, rendering the products less popular.

### SUMMARY OF THE INVENTION

In view of the foregoing, it is an objective of the invention to provide a simple but effective means to improve the humidification apparatus to solve the problem of water condensate and to reduce discomfort of the patient.

The invention provides a preheating device for the pipeline between an air pressure source and a humidifier. In order not for the intake duct to accumulate water due to condensation, a preheating device is installed in the pipeline between the air pressure source and the humidifier. The air entering the humidifier is preheated. By increasing the gas temperature and reducing the relativity humidity thereof, the air has a better ability to absorb water vapor. Within a short time, the air can fully mix with the water vapor generated by the heated water carrier of the humidifier. This prevents the problem of excessively outputting condensed water in the prior art.

### BRIEF DESCRIPTION OF THE DRAWINGS

These and other features, aspects and advantages of the invention will become apparent by reference to the following description and accompanying drawings which are given by way of illustration only, and thus are not limitative of the invention, and wherein:

FIG. 1 is a schematic view showing how a conventional humidification apparatus functions; and

FIG. 2 is a schematic view showing how the disclosed humidification apparatus functions.

### DETAILED DESCRIPTION OF THE INVENTION

The present invention will be apparent from the following detailed description, which proceeds with reference to the accompanying drawings, wherein the same references relate to the same elements.

Please refer to FIG. 2. A gas supply means, commonly called the air pressure source 1, is built in with a fan or compressor (not shown). The air generated by the air pressure source 1 is sent to a humidifier 3 via a pipeline 2. The humidifier includes a humidification chamber 31 and a heater plate 32. The heater plate 32 heats up the water carrier inside the humidification chamber 31, so that the gas provided by the air pressure source 1 carries away the water vapor from the water carrier. The humidified gas is sent via the intake duct 4 to the patient at the best breathing temperature and humidity. To reduce condensed water, a heating element 41 is provided inside the intake duct to maintain the temperature of the gas.

The invention is featured in that a heating element 21 is provided inside the pipeline 2 between the air pressure source 1 and the humidifier 3. The air entering the humidifier 3 is preheated. By increasing the gas temperature and reducing the relativity humidity thereof, the air has a better ability to absorb water vapor. Within a short time, the air can fully mix with the water vapor generated by the heated water carrier of the humidifier 3.

Although the above-mentioned design of preheating the air entering the humidifier is relatively simple, the effect is prominent. The invention has the following advantages:

1. The cost increase is very limited, rendering the product still competitive.

2. The preheated gas has a better ability in absorbing water vapor. Within a short time, the air can fully mix with the water vapor generated by the heated water carrier of the humidifier. This reduces the use of distilled water in the humidifier.

3. It solves the problem of water condensate because of excessive output of humidity by the humidifier.

4. Because of the improvement in condensation, the heating element in the intake duct can save more electrical power.

5. With the improvement in the condensation problem, it is not necessary to increase the heating power of the humidifier to compensate the loss in temperature and water vapor, thereby saving the use of distilled water and electrical power.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A preheating device of a pipeline between an air pressure source and a humidifier, comprising: an air pressure source, a humidifier, and an intake duct; wherein the air pressure source sends the air generated by a built-in fan or compressor inside a has supply means to the humidifier via a pipeline; the humidifier has a humidification chamber and a heater plate, the heater plate heating a water carrier in the humidification chamber so that the air provided by the air pressure source carries away the water vapor produced by the water carrier; the intake duct sends the humidified air to a patient; a heating element is provided in the pipeline between the air pressure source and the humidifier to increase the temperature of the gas entering the humidifier and to reduce the relative humidity thereof for the water vapor generated by the heated water carrier of the humidifier to fully mix with the gas in a short time, thereby reducing water condensate due to excessive output of humidity by the humidifier.
